(19) United States
(12) Patent Application Publication
(10) Pub. No.: US 2019/0343468 A1
(43) Pub. Date: Nov. 14, 2019

(54) RADIATION IMAGING APPARATUS AND IMAGING SYSTEM

(71) Applicant: CANON KABUSHIKI KAISHA, Tokyo (JP)

(72) Inventors: Keiichi Nomura, Kawasaki-shi (JP); Kazumi Nagano, Tokyo (JP); Tomoyuki Oike, Yokohama-shi (JP); Shinichi Takeda, Kawasaki-shi (JP)

(21) Appl. No.: 16/520,760

(22) Filed: Jul. 24, 2019

Related U.S. Application Data
(63) Continuation of application No. PCT/JP2017/042518, filed on Nov. 28, 2017.

(30) Foreign Application Priority Data
Feb. 8, 2017 (JP) 2017-021603

(51) Int. Cl. A61B 6/00; G01T 1/20; G01T 7/00; H04N 5/335

(52) U.S. Cl. CPC A61B 6/00; G01T 1/20; H04N 5/32; H04N 5/335; G01T 7/00

(57) ABSTRACT

One aspect of the invention is a radiation imaging apparatus, comprising a first imaging panel including a first sensor substrate including a center region and a peripheral region, and a first scintillator arranged in the center region, a second imaging panel including a second sensor substrate including a center region and a peripheral region, and a second scintillator arranged at the center region, the second imaging panel being arranged above the first imaging panel, a supporting base configured to support the first imaging panel upward, and a supporting member arranged below the peripheral region of the second sensor substrate so that a load acting on the peripheral region of the second sensor substrate downward is received by the supporting base.

### CROSS-REFERENCE TO RELATED APPLICATIONS

This application is a Continuation of International Patent Application No. PCT/JP2017/042518, filed Nov. 28, 2017, which claims the benefit of Japanese Patent Application No. 2017-021603, filed Feb. 8, 2017, both of which are hereby incorporated by reference herein in their entirety.

### BACKGROUND OF THE INVENTION

### Field of the Invention

The present invention relates to a radiation imaging apparatus and an imaging system and, more particularly, to a radiation imaging apparatus arranged such that a radiation image based on energy subtract processing can be obtained.

### Background Art

There are radiation imaging apparatuses that can perform processing for obtaining two image data for a single object (for example, a patient) and forming one radiation image based on the difference between these two image data. More specifically, the two image data are obtained at different radiation doses, and the difference between these two image data is obtained using a predetermined coefficient. This makes it possible to observe a desired target portion or change an observation target (for example, from an internal organ to a bone) by changing the coefficient. This image processing is called energy subtraction processing or simply subtraction processing or the like.

PTL 1 describes the structure of a radiation imaging apparatus including two imaging panels arranged parallel to each other. Each imaging panel includes a sensor substrate and a scintillator arranged at the center region. According to PTL 1, it is possible to obtain two image data at once with this structure.

### CITATION LIST

### Patent Literature

PTL 1: Japanese Patent Laid-Open No. 2016-156719

In some cases, a heavy load acts on a radiation imaging apparatus upon contact of an object to the radiation imaging apparatus, laying of the object on the radiation imaging apparatus, or the like. According to the structure of PTL 1, when a load acts on one of the two imaging panels on the object side, a stress is generated at its end portion. This causes damage to the end portion, and reliability of the radiation imaging apparatus may degrade in some cases.

It is an object of the present invention to provide a technique advantageous in improving reliability by improving durability against the load on the radiation imaging apparatus arranged so that a radiation image based on energy subtraction processing can be obtained.

### SUMMARY OF INVENTION

An aspect of the present invention relates to a radiation imaging apparatus. The radiation imaging apparatus comprises a first imaging panel including a first sensor substrate including a center region and a peripheral region and a first scintillator arranged in the center region, a second imaging panel including a second substrate including a center region and a peripheral region and a second scintillator arranged at the center region, the second imaging panel being arranged above the first imaging panel, a supporting base configured to support the first imaging panel upward, and a supporting member arranged below the peripheral region of the second sensor substrate so that a load acting on the peripheral region of the second sensor substrate downward is received by the supporting base.

Further features of the present invention will become apparent from the following description of exemplary embodiments with reference to the attached drawings.

### BRIEF DESCRIPTION OF THE DRAWINGS

The accompanying drawings, which are incorporated in and constitute a part of the specification, illustrate presently preferred embodiments of the invention, and together with the general description given above and the detailed description of the preferred embodiments given below, serve to explain the principles of the invention.

FIGS. 1A and 1B are views for explaining an example of the structure of a radiation imaging apparatus;

FIG. 2 is a view for explaining an example of the structure of an imaging panel;

FIGS. 3A and 3B are views for explaining another example of the structure of the radiation imaging apparatus;

FIGS. 4A, 4B, 4C, 4D, 4E, 4F, and 4G are views for explaining various modifications of the sectional structures of the radiation imaging apparatuses;

FIG. 5 is a view for explaining another example of the structure of a radiation imaging apparatus; and

FIG. 6 is a view for explaining an example of the arrangement of an imaging system.

### DESCRIPTION OF THE EMBODIMENTS

Preferred embodiments of the present invention will now be described with reference to the accompanying drawings. Note that the drawings are shown merely for the purpose of explaining structures or arrangements, and the dimensions of members shown in the drawings do not necessarily reflect the actuality. In addition, the same reference numerals denote the same members or the same constituent elements in the drawings, and a description of repetitive contents will be omitted below.

### First Embodiment

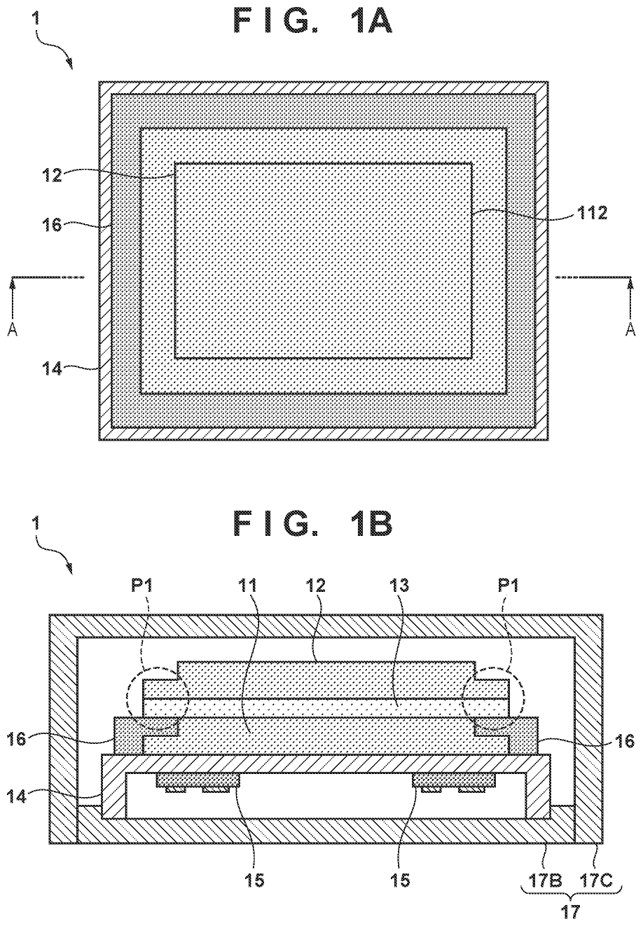

FIGS. 1A and 1B are schematic views showing the structure of a radiation imaging apparatus 1 according to the first embodiment. FIG. 1A is a plan view of the radiation imaging apparatus 1. FIG. 1B is a sectional view of the radiation imaging apparatus 1 along a cut line A-A. The radiation imaging apparatus 1 includes imaging panels 11 and 12, a filter member 13, a supporting base 14, a mounting substrate 15, a supporting member 16, and a housing 17 which contains the above components.

The housing 17 includes a bottom surface portion (a lower surface portion) 17B, and a cover portion 17C forming a top plate (an upper surface portion), and side walls. The housing 17 is made of a material having a relatively low radiation absorbance. Examples of the housing 17 are a plastic, carbon, or the like. A preferable material can be carbon fiber reinforced plastic (CFRP). Note that FIG. 1A does not illustrate the housing 17 in order to illustrate the above elements contained in the housing 17.

The supporting base 14 is fixed on the bottom surface portion 17B so as to form a space between the supporting base 14 and the bottom surface portion 17B. The imaging panels 11 and 12, the filter member 13, and the supporting member 16 are arranged on the supporting base 14. More specifically, the imaging panel 11 is supported by the supporting base 14 upward and fixed. The imaging panel 12 is arranged above the imaging panel 11. The filter member 13 can absorb part of radiation energy and is arranged between the imaging panel 11 and the imaging panel 12. An adhesive agent (not shown) is applied between the filter member 13 and the imaging panel 11 and between the filter member 13 and the imaging panel 12. These components are fixed to each other. The supporting member 16 will be described in detail later. In this embodiment, the supporting member 16 is arranged in the peripheral portion of the imaging panel 11.

The mounting substrate 15 is fixed in the space between the supporting base 14 and the bottom surface portion 17B. The imaging panels 11 and 12 are connected by a flexible wiring portion (not shown) for driving the imaging panels 11 and 12. An FPC (flexible printed circuit board), a COF (chip on film), or the like can be used for this wiring portion. The wiring portion extends from the mounting substrate 15 to the imaging panels 11 and 12 via an opening (not shown) formed in the side surface portion of the supporting base 14.

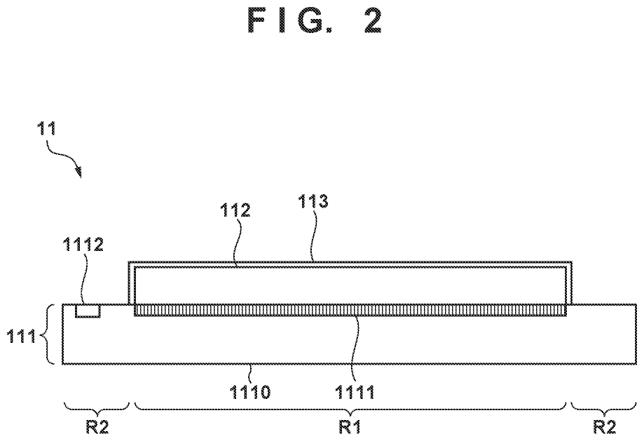

FIG. 2 is a schematic view showing the structure of the imaging panel 11. The imaging panel 11 includes a sensor substrate 111, a scintillator 112, and a protective film 113. The sensor substrate 111 includes a center region R1 and a peripheral region R2 thereof in a planar view (a planar view with respect to the imaging surface or its parallel plane of the imaging panel 11 in this specification). The sensor substrate 111 includes an insulating substrate 1110 made of an insulating material such as glass, a sensor array 1111 in which a plurality of sensors are arranged on the insulating substrate 1110, and a wiring connection portion 1112. The sensor array 1111 is positioned within the center region R1. A photoelectric conversion element (a PIN sensor, a MIS sensor or the like) made of amorphous silicon is used as each sensor.

The wiring connection portion 1112 is arranged in part of the peripheral region R2. The wiring connection portion 1112 serves as an external terminal (or it may be called an “electrode pad” or the like) for reading out a signal from the sensor array 1111 and is electrically connected to the above-mentioned wiring portion. In this embodiment, the imaging panel 11 (the insulating substrate 1110) is rectangular in the planar view. As the wiring connection portion 1112, a plurality of external terminals are typically arranged along the two adjacent sides (the two sides forming a corner) of the insulating substrate 1110.

The scintillator 112 is arranged in the center region R1 of the sensor substrate 111 so as to cover the sensor array 1111. The scintillator 112 converts the radiation entering the imaging panel 11 into light. This light is also called scintillation light and detected by the sensor substrate 111. A known phosphor material is used for the scintillator 112. Examples of the phosphor material are thallium-doped cesium iodide (Csl:T1), sodium-doped cesium iodide (Csl:Na), and gadolinium oxysulfide (Gd₂O₂S:Tb(GOS)).

The protective film 113 is made of a damp-proof material and arranged to cover the upper surface and the side surfaces of the scintillator 112, thereby preventing deliquescence of the scintillator 112. In this embodiment, the protective film 113 further has a light reflection property. This makes it possible to reflect the scintillation light toward the sensor substrate 111. The protective film 113 is made of, for example, polyparaxylene, a hot melt resin, aluminum, or a laminated sheet thereof.

The imaging panel 11 has a convex outer shape in a direction parallel to the imaging surface by the above structure. The step of the imaging panel 11 can be given by mainly the thickness (typically about 500 [μm] to 1 [mm] or more) of the scintillator 112. The imaging panel 12 also has the above structure (see FIG. 2), that is, includes the sensor substrate 111, the scintillator 112, and the protective film 113. Since the structure of the imaging panel 12 is the same as that of the imaging panel 11, a detailed description thereof will be omitted. Note that the imaging panels 11 and 12 need not have the above structure, but can have another known structure. For example, a sensor protective film and/or scintillator underlayer may be arranged between the sensor substrate 111 and the scintillator 112.

Referring back to FIG. 1B, in this embodiment, the imaging panels 11 and 12 are arranged such that the scintillator 112 is positioned on the upper side of the sensor substrate 111. The upper side in FIG. 1B is the radiation irradiation side, that is, the imaging panels 11 and 12 are used as a so-called front-side illumination type.

The radiation is emitted downward in a state in which the object (not shown) such as a patient is laid on the cover portion 17C of the housing 17. The radiation passing through the object and the cover member 17C is detected by the imaging panel 12. The filter member 13 is a K-terminal filter made of a metal material such as copper (Cu) and absorbs the low energy component of the radiation passing through the imaging panel 12. More specifically, the filter member 13 absorbs the low energy component of the K absorption end of the radiation passing through the imaging panel 12. The radiation passing through the filter member 13 is detected by the imaging panel 11.

That is, the upper imaging panel 12 with respect to the filter member 13 performs imaging based on the radiation having relatively small energy. The lower imaging panel 11 with respect to the filter member 13 performs imaging based on the radiation having relatively large energy. Therefore, two image data can be obtained by one radiation imaging.

With the above structure, the image data obtained from the imaging panel 11 and the image data obtained from the imaging panel 12 represent pieces of image information of the single object, but the data values (signal values) of these pieces of information are different from each other. The energy subtraction processing can be used using these two image data. More specifically, arithmetic processing is performed for these two image data using a predetermined coefficient to allow observation of the examination target portion. By changing the coefficient, the observation target can be changed to another portion (for example, from an internal organ to a bone).

Note that the filter member 13 may be omitted as another embodiment because the radiation is attenuated while passing through the imaging panel 12. Alternatively, as still another embodiment, the insulating substrate 1110 of the imaging panel 12 may be arranged to also serve as the filter member 13.

Assume that the object lies on the cover portion 17C and the object changes the posture on the cover portion 17C. In this case, if the top plate of the cover portion 17C is deformed, a load acts on the imaging panel 12 downward. In this embodiment, the supporting member 16 is arranged in the peripheral portion of the imaging panel 11 on the supporting base 14, and the downward load of the imaging panel 12 is supported by the supporting base 14. This will be described below with reference to FIG. 1B.

If the supporting member 16 is not arranged, a portion P1 illustrated in FIG. 1B, that is, the end portion (more specifically the peripheral region R2 of the sensor substrate 111) of the imaging panel 12 and the end portion of the filter member 13 cannot stand the downward load and may be demanded. Note that a damping material such as sponge may be arranged between the top plate of the cover portion 17C and the imaging panel 12. Damage may occur even in this arrangement. In this embodiment, the supporting member 16 supports the peripheral region R2 of the sensor substrate 111 of the imaging panel 12 upward and is arranged to cause the supporting base 14 to support the downward load acting on the peripheral region R2.

The outer edge of the imaging panel 11 and the outer edge of the imaging panel 12 are located inside the outer edge of the supporting base 14. In addition, the filter member 13 is arranged such that this outer edge matches the outer edge of the imaging panel 12. In this structure, the supporting member 16 is arranged to extend up to the supporting base 14 while filling the space between the imaging panel 11 and the filter member 13. The supporting member 16 contacts the upper surface of the supporting base 14 and is fixed thereto. Referring to FIG. 1A, the supporting member 16 is annularly arranged along the outer edge of the imaging panel 11 in the planar view. In this embodiment, the supporting member 16 is integrally formed annularly, but the supporting member 16 may be discretely arranged as another embodiment.

According to this embodiment, part of the load acting on the portion P1 is supported by the supporting base 14 (the load is appropriately transmitted to the supporting base 14) by extending the supporting member 16 up to the supporting base 14 while the supporting member 16 fills the space between the imaging panel 11 and the filter member 13. The remaining part of the load is supported by the supporting base 14 (the load is appropriately transmitted to the supporting base 14) via the sensor substrate 111 (the peripheral region R2 of the sensor substrate 111) of the imaging panel 11 while the supporting member 16 fills the above space.

The supporting member 16 is made of an insulating material. The supporting member 16 is arranged to set its rigidity to be higher than that of the scintillator 112 so that the scintillator 112 will not be damaged by the above load. For example, a material containing at least one of a phenol resin, epoxy resin, silicone resin, acrylic resin, polyether ether ketone (PEEK) resin, fluoroplastic, and urethane resin can be used for the supporting member 16. A thermosetting resin, ultraviolet curing resin, or the like can be used for the supporting member 16 so as to form it in a desired shape. Since the supporting member 16 includes a portion adjacent to the wiring connection portion 1112 and the wiring portion connected thereto, an antistatic material such as polyethylene terephthalate, vinyl chloride, or polycarbonate is used for the supporting member 16. A material not containing chlorine is preferably used for the supporting member 16 to prevent corrosion of the wiring connection portion and the wiring portion.

According to this embodiment, a stress acting on the portion P1 can be relaxed, and damage to the imaging panel 12 can be prevented. Therefore, according to this embodiment, the durability (strength) against the above load can be improved, and the reliability of the radiation imaging apparatus 1 can be improved.

From the viewpoint of prevention of damage to the end portion of the imaging panel 12, the filter member 13 supports upward the end portion of the imaging panel 12 together with the supporting member 16. The filter member 13 can be expressed to play a role of part of the function for supporting this end portion. In other words, according to this embodiment, the supporting member 16 supports upward the end portion of the imaging panel 12 together with the filter member 13.

### Second Embodiment

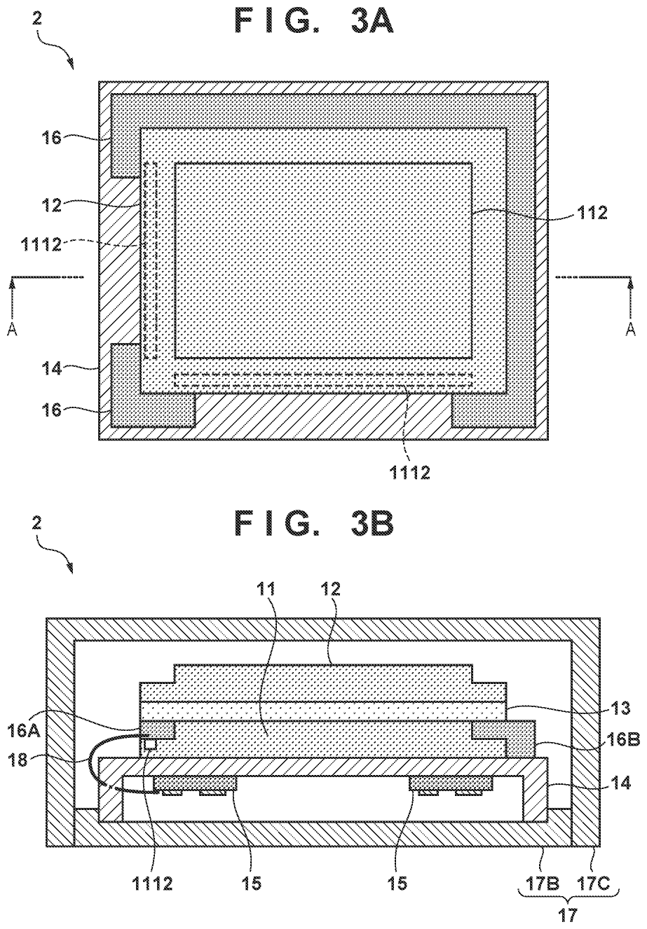

The first embodiment has described the structure in which the supporting member 16 extends up to the supporting base 14 while filling the space between the imaging panel 11 and the filter member 13, thereby allowing the supporting base 14 to receive the load acting on the imaging panel 12 downward. The second embodiment is mainly different from the first embodiment in that parts of a supporting member 16 do not extend up to a supporting base 14. FIGS. 3A and 3B are schematic views showing the structure of a radiation imaging apparatus 2 of this embodiment in the same manner as in FIGS. 1A and 1B (see the first embodiment). The supporting member 16 is arranged to extend to the supporting base 14 at the upper side and the right side in FIG. 3A and not to extend to the supporting base 14 at the left side and the lower side.

As has been described with reference to FIG. 2, wiring connection portions 1112 are typically arranged along two adjacent sides of an insulating substrate 1110. For example, the sensor substrate 111 further includes a driving unit (for example, a vertical scanning circuit) for driving the pixels for each row of the sensor array 1111 and a signal readout unit (for example, a horizontal scanning circuit) for reading out signals for each column from the sensor array 1111. The driving unit and the signal readout unit are not illustrated, but arranged in each of the left side and the lower side of the insulating substrate 1110 of an imaging panel 11. In correspondence with these, wiring connection portions 1112 are arranged along the left side and the lower side of the insulating substrate 1110. In FIG. 3A, the wiring connection portions 1112 for the imaging panel 11 are illustrated by broken lines, respectively. In FIG. 3B, for the sake of descriptive simplicity, out of the supporting member 16, a portion which covers the wiring connection portions 1112 is indicated by a “portion 16A”, and a portion which does not cover the wiring connection portions 1112 is indicated by a “portion 16B”.

As can be obvious from FIGS. 3A and 3B, the portion 16A which covers the wiring connection portion 1112 out of the supporting member 16 is arranged not to extend up to the supporting base 14. The wiring connection portions 1112 are connected to a mounting substrate 15 by a flexible wiring portion 18. According to this embodiment, since the portion 16A of the supporting member 16 does not extend up to the supporting base 14, the wiring portion 18 can easily extend from the wiring connection portions 1112 to the mounting substrate 15.

According to this embodiment, since the portion 16A of the supporting member 16 sufficiently fills the space between the imaging panel 11 and a filter member 13, the above load is supported by the supporting base 14 via the sensor substrate 111 of the imaging panel 11. According to this embodiment, in addition to the same effect as in the first embodiment, the arrangement of the wiring portion 18 can be easily implemented depending on the positions of the wiring connection portions 1112 in the structure including the supporting member 16. This structure can cope with various arrangements.

Note that in this embodiment, the portion 16A exemplifies a mode not to extend up to the supporting base 14 depending on the positions of the wiring connection portion 1112. However, the portions 16A and 16B may be selectively arranged in accordance with another purpose or the like.

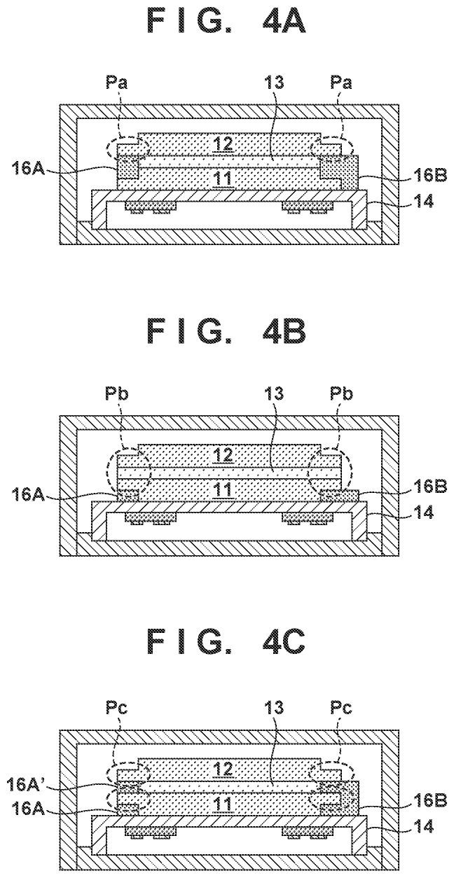

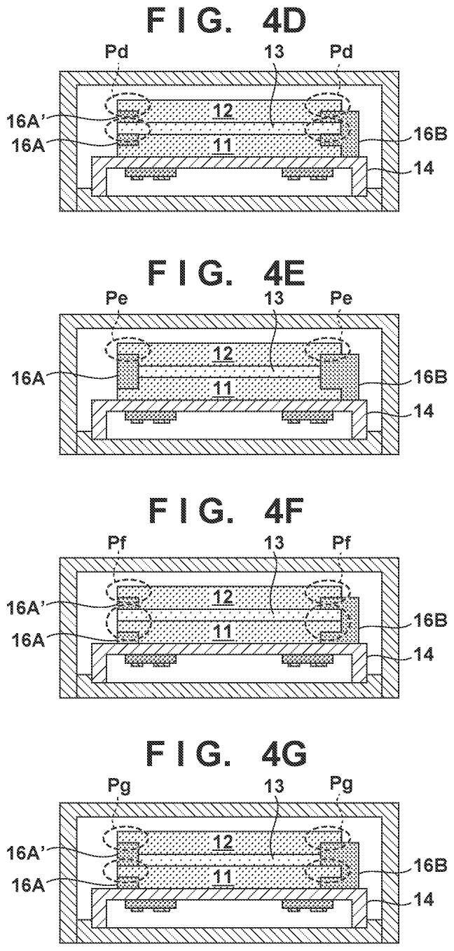

FIGS. 4A to 4G are schematic views for explaining various modifications of the second embodiment. Even in these modifications, the same effect as in the second embodiment can be obtained. Note that for the sake of illustrative simplicity, the wiring portion 18 and the wiring connection portions 1112 are not illustrated.

An example of FIG. 4A is different from the structure (the structure in FIG. 3B) of the second embodiment in that the filter member 13 is arranged inside the outer edges of the sensor substrates 111 of the imaging panels 11 and 12. In this example, although the filter member 13 is illustrated such that its outer edge almost matches the outer edge of the scintillator 112 of the imaging panel 11. However, the outer edge of the filter member 13 may be arranged outside the outer edge of the scintillator 112. In order to limit the radiation energy to be detected by the imaging panel 11, the outer edge of the filter member 13 is made to almost match the outer edge of the scintillator 112 or is arranged outside the outer edge of the scintillator 112.

In the example of FIG. 4A, in order to prevent damage to a portion Pa in FIG. 4A, that is, the end portion of the imaging panel 12 (a peripheral region R2 of the sensor substrate 111, and this region will be simply referred to as an “end portion”), the supporting member 16 is arranged to support the portion Pa upward and the supporting base 14 receives the load acting on the portion Pa downward. More specifically, the portion 16A of the supporting member 16 is arranged to fill the region between the end portion of the imaging panel 11 and the end portion of an imaging panel 12 so as to cover the side surfaces of the filter member 13. On the side opposite to the portion 16A, the portion 16B of the supporting member 16 extends up to the supporting base 14 so as to fill the region between the end portion of the imaging panel 11 and the end portion of the imaging panel 12 while covering the side surfaces of the filter member 13.

According to the example of FIG. 4A, in addition to the same effect as in the second embodiment, the supporting member 16 is arranged to cover the side surfaces of the filter member 13. The positional shift of the filter member 13 in the horizontal direction (a direction parallel to the imaging surface) and the scratch of the imaging panels 11 and 12 upon the positional shift can also be prevented.

An example of FIG. 4B is different from the arrangement of the second embodiment in that the scintillator 112 of the imaging panel 11 is arranged below the sensor substrate 111, that is, the imaging panel 11 is a back-side illumination type. In the example of FIG. 4B, since no space is formed between the imaging panel 11 and the imaging panel 12, the supporting member 16 is arranged on the supporting base 14 near below the imaging panel 11. More specifically, the portions 16A and 16B of the supporting member 16 are arranged to fill the region between the end portion of the imaging panel 11 and the supporting base 14. Accordingly, damage to a portion Pb shown in FIG. 4B, that is, the end portions of the imaging panels 11 and 12 can be appropriately prevented, and damage to the end portion of the filter member 13 can be appropriately prevented.

As shown in FIG. 4C, the filter member 13 can be arranged inside the outer edges of the sensor substrates 111 of the imaging panels 11 and 12 as in the example of FIG. 4A. In the example of FIG. 4C, the portion 16A of the supporting member 16 is arranged to fill the region between the end portion of the imaging panel 11 and the supporting base 14. In this example, the supporting member 16 further includes a portion 16A′ which fills the region between the end portion of the imaging panel 11 and the end portion of the imaging panel 12 and covers the side surfaces of the filter member 13. The portion 16B on the side opposite to the portion 16A extends up to the supporting base 14 so that the portion 16B integrally fills regions from the region between the end portion of the imaging panel 11 and the end portion of the imaging panel 12 to the region between the end portion of the imaging panel 11 and the supporting base 14 while covering the side surfaces of the filter member 13. With this structure, damage to a portion Pc shown in FIG. 4C, that is, the end portions of the imaging panels 11 and 12 can be appropriately prevented, and the positional shift of the filter member 13 can also be prevented.

An example of FIG. 4D is different from the second embodiment in that the imaging panel 11 is used as a front-side illumination type and the imaging panel 12 is used as a back-side illumination type. In the example of FIG. 4D, the portion 16A of the supporting member 16 is arranged to fill the region between the end portion of the imaging panel 11 and the end portion of the filter member 13. In this example, the supporting member 16 further includes the portion 16A′ which fill the region between the end portion of the imaging panel 12 and the end portion of the filter member 13. The portion 16B on the side opposite to the portion 16A extends up to the supporting base 14 while integrally filling the region from the region between the end portion of the imaging panel 12 and the end portion of the filter member 13 to the region between the end portion of the imaging panel 11 and the end portion of the filter member 13. With this structure, damage to a portion Pd shown in FIG. 4D, that is, the end portion of the imaging panel 12 and the end portion of the filter member 13 can be appropriately prevented.

As exemplified in FIG. 4E, the filter member 13 may be positioned inside the outer edges of the sensor substrates 111 of the imaging panels 11 and 12. In the example of FIG. 4E, the portion 16A of the supporting member 16 is arranged to fill the region between the end portion of the imaging panel 11 and the end portion of the imaging panel 12 and cover the side surfaces of the filter member 13. In addition, the portion 16B on the side opposite to the portion 16A extends up to the supporting base 14 so as to fill the region between the end portion of the imaging panel 11 and the end portion of the imaging panel 12 while covering the side surfaces of the filter member 13. With this structure, damage to a portion Pe shown in FIG. 4E, that is, the end portion of the imaging panel 12 can be appropriately prevented, and the positional shift of the filter member 13 can be prevented.

An example of FIG. 4F is mainly different from the second embodiment in that the imaging panels 11 and 12 are of a back-side illumination type. In the example of FIG. 4F, the portion 16A of the supporting member 16 is arranged to fill the region between the end portion of the imaging panel 11 and the supporting base 14. In this embodiment, the supporting member 16 further includes a portion 16A′ which fills the region between the end portion of the imaging panel 12 and the end portion of the filter member 13. In addition, the portion 16B on the side opposite to the portion 16A extends up to the supporting base 14 so as to integrally fill the regions from the region between the end portion of the imaging panel 12 and the end portion of the filter member 13 to the region between the end portion of the imaging panel 11 and the supporting base 14. With this structure, damage to a portion Pf shown in FIG. 4F, that is, the end portions of the imaging panels 11 and 12 and the end portion of the filter member 13 can be appropriately prevented.

As exemplified in FIG. 4G, the filter member 13 is arranged inside the outer edges of the sensor substrates 111 of the imaging panels 11 and 12. In the example in FIG. 4G, the portion 16A of the supporting member 16 is arranged so as to fill the region between the end portion of the imaging panel 11 and the supporting base 14. In this example, the supporting member 16 further includes the portion 16A′ which fills the region between the end portion of the imaging panel 11 and the end portion of the imaging panel 12 while covering the side surfaces of the filter member 13. The portion 16B on the side opposite to the portion 16A extends up to the supporting base 14 so as to integrally fill the regions from the region between the end portion of the imaging panel 11 and the end portion of the imaging panel 12 to the region between the end portion of the imaging panel 11 and the supporting base 14 while covering the side surfaces of the filter member 13. With this structure, damage to a portion Pg shown in FIG. 4G, that is, the end portions of the imaging panels 11 and 12 can be appropriately prevented, and the positional shift of the filter member 13 can be prevented.

As still another modification, the portion 16B of the supporting member 16 covers the side surfaces of the sensor substrate 111 of the imaging panel 11 and extends up to the imaging panel 12 to further cover the side surfaces of the sensor substrate 111 of the imaging panel 12. For example, dicing cracks can be formed in the side surface (cutting surface) of the insulating substrate 1110 of the sensor substrate 111. By covering this side surface, intrusion of water, a chemical solution, or the like to the insulating substrate 1110 during the manufacture can be prevented. Accordingly, the product life of the radiation imaging apparatus 2 can be prolonged, and its reliability can be improved.

### Third Embodiment

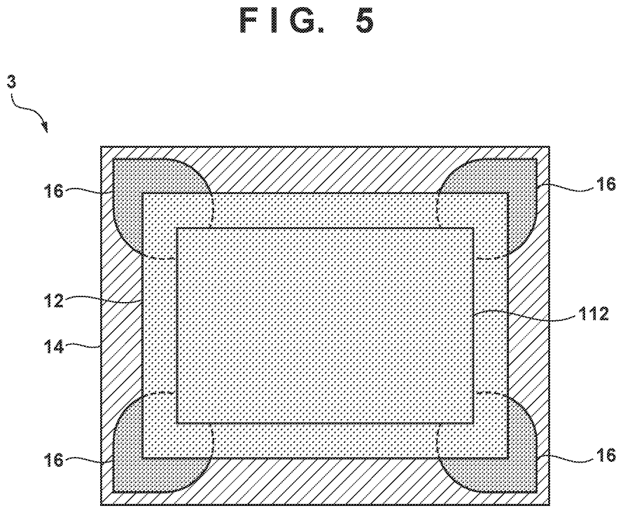

FIG. 5 is a plan view of a radiation imaging apparatus 3 according to the third embodiment. As described above, imaging panels 11 and 12 have a rectangular shape in the planar view. The first embodiment described above has exemplified the structure in which the supporting member 16 is arranged annularly along the outer edge of the imaging panel 11 in the planar view. In the third embodiment, a supporting member 16 is arranged at the corner portion of the imaging panel 11. In general, if the imaging panels 11 and 12 are rectangular, each corner portion of a sensor substrate 111 is readily damaged most. For this reason, in this embodiment, the supporting member 16 is arranged at this corner portion.

According to this embodiment, the corner portion of the sensor substrate 111 at which the strength tends to lower can be reinforced, and a wiring connection portion 1112 arranged along the side of the sensor substrate 111 can be exposed. The connection or reconnection (repair or replacement of a connection portion 18) of the wiring portion 18 can be easily performed. As still another embodiment, the supporting member 16 may be arranged so that the above corner portion is arranged as in FIG. 5, and the supporting member 16 is made not to extend up to a supporting base 14, that is, the side portion except the corner portion as in the portion 16A (see FIG. 3B)

(Imaging System)

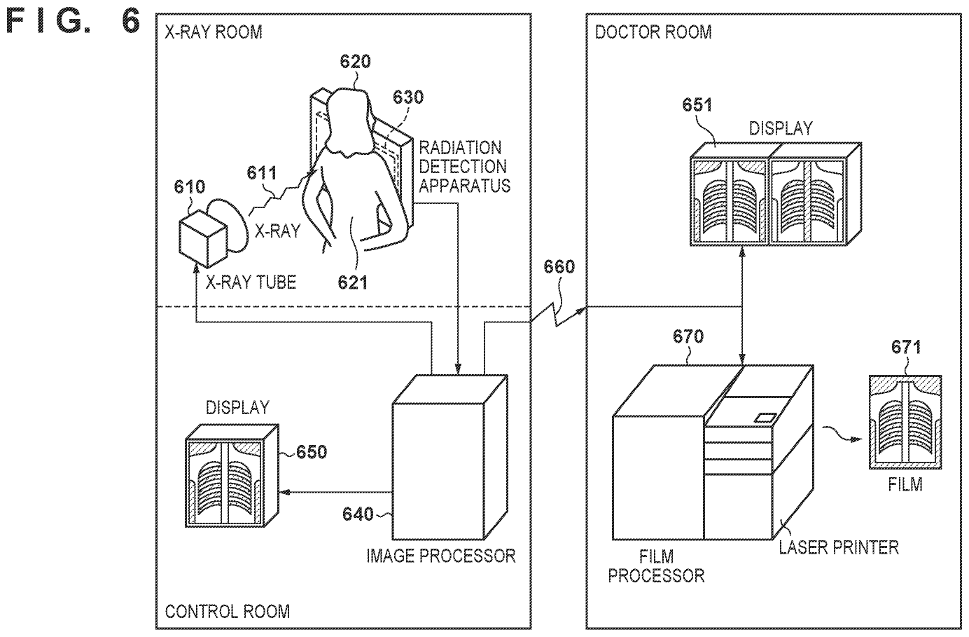

As exemplified in FIG. 6, the radiation imaging apparatus 1 or 2 described in each embodiment described above can be applied to an imaging system which performs so-called X-ray imaging. X-rays are typically used as the radiation, but alpha-rays, beta-rays, or the like can be used. X-rays 611 generated by an X-ray tube 610 (radiation source) pass through a chest portion 621 of an object 620 such as a patient and enter a radiation imaging apparatus 630. The X-rays 611 entering the apparatus 630 contain in-vivo information of the object 620, thereby obtaining electrical information corresponding to the X-rays 611 entering the apparatus 630. This electrical information is converted into a digital signal and undergoes predetermined signal processing by, for example, a processor 640.

A user such as a doctor can observe the radiation image corresponding to this electrical information on, for example, a display 650 (display unit) of a control room. The user can transfer the radiation image or its data to a remote place by a predetermined communication unit 660. This radiation image can be observed on a display 651 of a doctor room as another place. In addition, the user can record this radiation image or its data in a predetermined recording medium such as a film 671 using a processor 670.

(Others)

Several preferred embodiments have been described above. However, the present invention is not limited to these examples and may partially be modified without departing from the scope of the invention. For example, other elements may be combined with the contents of the embodiments in accordance with the object, application purpose, and the like, and the contents of a certain embodiment may be combined with part of the contents of another embodiment. In addition, individual terms described in this specification are merely used for the purpose of explaining the present invention, and the present invention is not limited to the strict meanings of the terms and can also incorporate their equivalents.

According to the present invention, the reliability of the radiation imaging apparatus can be improved.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A radiation imaging apparatus comprising:
a first imaging panel including a first sensor substrate including a center region and a peripheral region, and a first scintillator arranged in the center region;
a second imaging panel including a second sensor substrate including a center region and a peripheral region, and a second scintillator arranged at the center region, the second imaging panel being arranged above the first imaging panel;
a supporting base configured to support the first imaging panel upward; and
a supporting member arranged below the peripheral region of the second sensor substrate so that a load acting on the peripheral region of the second sensor substrate downward is received by the supporting base.

2. The radiation imaging apparatus according to claim 1, wherein the supporting member is arranged outside an outer edge of the first scintillator in a planar view with respect to an imaging surface of the first imaging panel.

3. The radiation imaging apparatus according to claim 2, wherein
in the planar view, an outer edge of the first imaging panel and an outer edge of the second imaging panel are arranged inside an outer edge of the supporting base, and
at least part of the supporting member extends outside an outer edge of the first sensor substrate in the planar view and contacts the supporting base.

4. The radiation imaging apparatus according to claim 2, wherein
in the planar view, an outer edge of the first imaging panel and an outer edge of the second imaging panel are arranged inside an outer edge of the supporting base,
the first sensor substrate further includes a wiring connection portion arranged in part of the peripheral region,
the supporting member includes a first portion configured to cover the wiring connection portion and a second portion different from the first portion, and
the second portion out of the first portion and the second portion extends outside an outer edge of the first sensor substrate in the planar view and contacts the supporting base.

5. The radiation imaging apparatus according to claim 1, wherein the first imaging panel and the second imaging panel are arranged to position the first scintillator and the second sensor substrate between the first sensor substrate and the second scintillator.

6. The radiation imaging apparatus according to claim 5, wherein the supporting member is arranged to fill a region between the peripheral region of the first sensor substrate and the peripheral region of the second sensor substrate.

7. The radiation imaging apparatus according to claim 1, wherein the first imaging panel and the second imaging panel are arranged to position the first sensor substrate and the second sensor substrate between the first scintillator and the second scintillator.

8. The radiation imaging apparatus according to claim 7, wherein the supporting member is arranged to fill a region between the peripheral region of the first sensor substrate and the supporting base or fill a region between the peripheral region of the first sensor substrate and the supporting base and a region between the peripheral region of the first sensor substrate and the peripheral region of the second sensor substrate.

9. The radiation imaging apparatus according to claim 1, wherein the first imaging panel and the second imaging panel are arranged to position the first scintillator and the second scintillator between the first sensor substrate and the second sensor substrate.

10. The radiation imaging apparatus according to claim 9, wherein the supporting member is arranged to fill a region between the peripheral region of the first sensor substrate and the peripheral region of the second sensor substrate.

11. The radiation imaging apparatus according to claim 1, wherein the first imaging panel and the second imaging panel are arranged to position the first sensor substrate and the second scintillator between the first scintillator and the second sensor substrate.

12. The radiation imaging apparatus according to claim 11, wherein the supporting member is arranged to fill a region between the peripheral region of the first sensor substrate and the supporting base and a region between the peripheral region of the first sensor substrate and the peripheral region of the second sensor substrate.

13. The radiation imaging apparatus according to claim 1, wherein the supporting member is arranged so as to cover at least part of a side surface of the first sensor substrate.

14. The radiation imaging apparatus according to claim 13, wherein the supporting member is arranged so as to cover at least part of a side surface of the second sensor substrate.

15. The radiation imaging apparatus according to claim 1, wherein the supporting member is made of a material containing at least one of a phenol resin, an epoxy region, a silicone resin, an acrylic resin, a polyether ether ketone (PEEK) resin, a fluoroplastic, and a urethane resin.

16. The radiation imaging apparatus according to claim 1, wherein in a planar view with respect to an imaging surface of the second imaging panel, the supporting member is arranged annularly along an outer edge of the second imaging panel.

17. The radiation imaging apparatus according to claim 1, wherein in a planar view with respect to an imaging surface of the second imaging panel, the second imaging panel is rectangular, and the supporting member is arranged at each corner portion of the second imaging panel.

18. The radiation imaging apparatus according to claim 1, further comprising a filter member arranged between the first imaging panel and the second imaging panel and configured to absorb part of radiation passing through the second imaging panel,
wherein the supporting member is arranged to cover at least part of a side surface of the filter member.

19. The radiation imaging apparatus according to claim 1, further comprising a housing configured to contain the first imaging panel, the second imaging panel, the supporting base, and the supporting member,
wherein the supporting base is fixed to a bottom surface portion of the housing.

20. An imaging system comprising:
a radiation imaging apparatus defined in claim 1; and
a radiation source configured to generate radiation.
